(19) United States
(12) Patent Application Publication
(10) Pub. No.: US 2018/0333103 A1
(43) Pub. Date: Nov. 22, 2018

(54) Algorithmic Approach for Estimation of Respiration and Heart Rates

(71) Applicant: One Health Group, LLC, Chantilly, VA (US)

(72) Inventors: Raghed El Bardan, Herndon, VA (US); Dhaval Malaviya, Herndon, VA (US); Albert Di Rienzo, Herndon, VA (US)

(21) Appl. No.: 15/982,890

(22) Filed: May 17, 2018

Related U.S. Application Data
(60) Provisional application No. 62/508,247, filed on May 18, 2017.

(51) Int. Cl. A61B 5/00; A61B 5/0205

(52) U.S. Cl. CPC A61B 5/7225; A61B 5/7257; A61B 5/7289; A61B 5/0002; A61B 5/02438; A61B 5/6822; A61B 5/7278; A61B 2503/40; A61B 5/0205

(57) ABSTRACT

One or more aspects of the disclosure relate to monitoring a resting subject's respiration and heart rate through an impulse radio ultra-wideband (IR-UWB) radar in a noninvasive fashion, in order to infer the corresponding health status including heart rate and respiration rate. Any other reflectance technology may be used. These two cardiopulmonary vital signs are derived based on the processing of recorded waveforms that are collected by the IR-UWB radar or any other reflectance technology system, after getting reflected-off the resting subject's body or relevant body part. A novel algorithm that processes the recorded waveforms is proposed to extract these vitals' signals and, accordingly, estimate their rates. This algorithm includes at least three major stages: i) noise reduction, ii) respiration rate estimation, and iii) heart rate estimation. Furthermore, the algorithm addresses the effects of harmonics and intermodulation between the breathing and heartbeat signals without requiring the implementation of filters.

### CROSS-REFERENCE TO RELATED APPLICATION

This application claims the benefit under 35 U.S.C. 119(e) of U.S. Provisional Patent Application No. 62/508,247, filed May 18, 2017, which is incorporated by reference herein.

### BACKGROUND

In 2002, the Federal Communications Commission (FCC) authorized the unlicensed use of ultra-wideband (UWB) technology in the frequency range from 3.1 to 10.6 GHz (ET Docket 98-153, First Report and Order 02-48), using an adequate wideband signal format with a low equivalent isotropically radiated power (EIRP) level (−41.3 dBm/MHz). Since then, UWB technology has attracted growing interest across many different verticals and fields, e.g., wireless communications and a diverse set of radar sensor applications.

UWB systems can be categorized into two classes: i) multi-band orthogonal frequency division multiplexing (MB-OFDM) UWB, and ii) impulse radio UWB (IR-UWB). The former class is primarily used for applications that support exceedingly high data rates such as video streaming, and is beyond the scope of this work. Note that this class is not compliant with energy-constrained applications, given that high performance electronics are required to operate an MB-OFDM radio. On the other hand, IR-UWB can be purposed to accommodate low-power consumption and low-complexity. Furthermore, an IR-UWB radar is characterized by: i) higher penetration capabilities, ii) robustness to interference and multipath, and iii) high precision ranging. The aforementioned characteristics of the latter class have motivated both the research community and the industry to explore using IR-UWB radars in energy-constrained, short-range wireless health applications.

In applications that are pertinent to the medical field, noninvasive measurement of vital sign parameters may be useful and helpful, in terms of detecting early signs of an illness or a disease, and preventing potential health risks that are tightly coupled with these parameters.

### SUMMARY

The following summary presents a simplified description of certain features. The summary is not an extensive overview and is not intended to identify key or critical elements.

One or more embodiments may include continuous monitoring of a resting subject's respiration and heart rates in a noninvasive fashion using an IR-UWB radar or any other reflectance technology system. Accordingly, an inference on the health status of this subject can be made. For example, one or more embodiments may use the knowledge of this inference in detecting/predicting early signs of an illness or a disease and, hence, preventing a complete onset of it. One or more cardiopulmonary vital signs may be inferred based on the processing of recorded waveforms that are collected by the IR-UWB radar or any other reflectance technology system, after being reflected-off the subject's body. An algorithm may process the recorded waveforms in order to extract these vitals. This algorithm may include three phases. A first phase may include noise reduction. A second phase may include estimating respiration rate. A third phase may include estimating heart rate. One or more phases may include utilizing tools and techniques from signal processing and logic analysis. Furthermore, the proposed algorithm may suppress the effects of harmonics and intermodulation between the breathing and heartbeat signals of the corresponding rates without requiring the implementation of filters.

One or more aspects of the disclosed process may be implemented in hardware devices or in a general purpose computer programmed with instructions based on the described algorithm.

Other technical features may be readily apparent to one skilled in the art from the following figures, descriptions, and claims.

### BRIEF DESCRIPTION OF DRAWINGS

The present disclosure is illustrated by way of example and not limited in the accompanying figures in which like reference numerals indicate similar elements and in which:

FIG. 1 is an illustrative example of a monostatic radar on the neck of an animal in accordance with one or more embodiments described herein;

FIG. 2 is an illustrative example of a bistatic radar on the neck of an animal in accordance with one or more embodiments described herein;

FIG. 3A is a first illustrative example of a multistatic radar on the neck of an animal in accordance with one or more embodiments described herein; FIG. 3B shows signal paths for the multistatic radar of FIG. 3A;

FIG. 4A is a second illustrative example of a multistatic radar on the neck of an animal in accordance with one or more embodiments described herein; FIG. 4B shows signal paths for the multistatic radar of FIG. 4A;

FIG. 5A shows an illustrative example of a multistatic radar on a narrow neck of an animal in accordance with one or more embodiments described herein; FIG. 5B shows signal paths for the multistatic radar of FIG. 5A;

FIG. 6 shows an illustrative example of a UWB sensor in accordance with one or more embodiments described herein;

FIG. 7 depicts a graphic of the reflectance technology application in accordance with one or more embodiments described herein; and

FIG. 8 depicts an illustrative block diagram of stages of a process in accordance with one or more embodiments described herein.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. It is to be understood that other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

The following description relates to configurations of ultra-wideband (UWB) sensors for obtaining physiological information from mammals. Specifically, aspects of the disclosure pertain to the use of UWB sensors as medical radar to the extent they use very low power Ultra Wide Band (UWB) radio frequency (RF) energy. In practice, the UWB medical radar emits a narrow pulse of electromagnetic energy, which propagates into a body. As the energy enters the body, small amounts of the incident energy are reflected back to the device. The reflections are due to the differences in dielectric properties of the illuminated tissues and organs. The reflected energy is then received and processed using application-specific signal processing algorithms to extract information on the type, location, size, and motion of the illuminated tissues and organs. It is appreciated that the greater the dielectric constant between illuminated tissues and organs increases the reflection (or backscatter) of the electromagnetic pulse.

Examples of UWB medical radar systems are found, for instance, in U.S. Pat. No. 7,725,150 to Tupin, Jr. et al. and U.S. Pat. No. 8,463,361 to Tupin, Jr., both assigned to LifeWave, Inc. of Los Altos, Calif., whose contents are expressly incorporated by reference to their entirety.

Ultra-wideband radar overcomes one of the limitations found with Doppler radar because of the extremely fine radial resolution (<5 mm) inherent with UWB radar, allowing the UWB sensor to more easily isolate different physiological processes based on their unique locations within the patient. The sensor can focus on one or more depths using traditional range sweep techniques and, if the sensor is configured as an array, further focal processing techniques based on beam-steering and beam-forming can be applied.

A contact-based UWB medical sensor for monitoring the health of animals has several distinct advantages over Doppler and off-body monitoring. The UWB radar does not need direct skin contact or coupling gels, allowing it to collect useful physiological data through fur or feathers yet by maintaining contact with fur or feathers. As such, the large reflective losses associated with the skin-air interface are significantly reduced. Second, assuming the electronics are sufficiently protected from the environment (e.g., sealed against rain and moisture or otherwise moisture resistant), the radar may operate when wet or dirty.

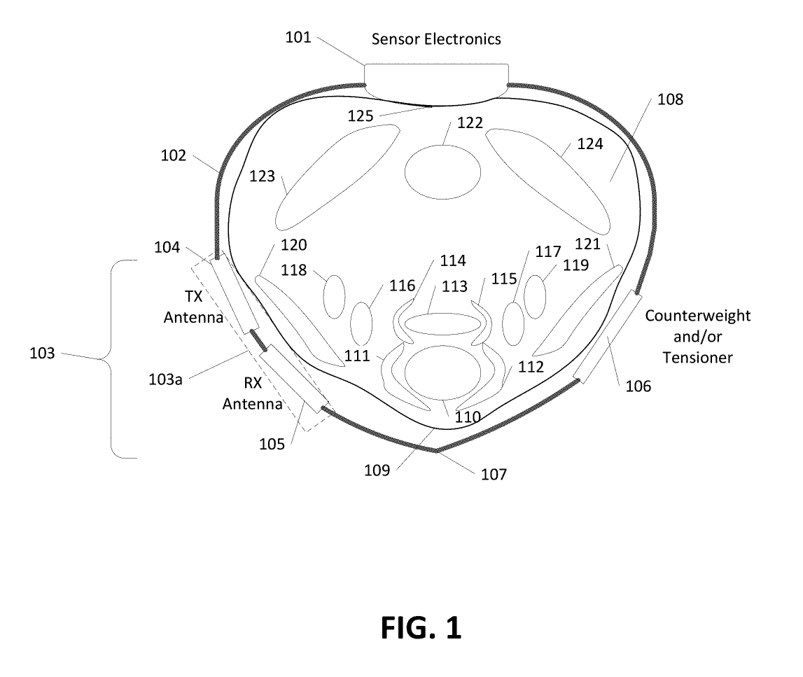

For instance, a UWB radar system may be placed on an animal's collar as shown in FIG. 1. FIG. 1 shows a cross-sectional view of an animal's neck and the collar being worn by the animal. The UWB radar system of FIG. 1 includes sensor electronics 101 in a housing and antennas 103 including a transmit antenna 104 and a receive antenna 105. The antennas 104 and 105 may be placed together in a singular housing 103a or may be housed separately. One advantage of housing the antennas 104 and 105 together is that the direction each antenna faces may be fixed with respect to the other antenna.

These components of the UWB radar system may be co-located at a single location or may be placed around the collar 102 as shown in FIG. 1 and connected by wires, cables, traces on a circuit board (rigid or flexible), or other known electrical connecting techniques. If co-located, an example of a size of the combination of the antennas 104 and 105 and the sensor electronics 101 may be 7.4 cm×2.3 cm×1.8 cm and weigh 29 g.

The UWB radar system monitors movement of different structures based on their different dielectric constants relative to surrounding structures or tissues. The change in location of the interfaces between these structures is monitored by the UWB radar system and is subsequently analyzed through known analysis techniques of UWB radar signals.

Aspects of this disclosure relate to configurations of the UWB radar system to provide improved signals for analysis. For reference, FIG. 1 shows the animal's neck 108 with skin 109, trachea 110 with surrounding muscles 111, 112, esophagus 113 with surrounding muscles 114, 115, carotid arteries 116, 117, jugular veins 118, 119, spinal column 122, and various other muscles (including lower muscle groups 120, 121, and upper muscle groups 123 and 124).

In one example, the UWB radar system with sensor electronics 101 and antennas 103 may be co-located (namely, the sensor electronics 101 module being positioned radially outward from antennas 103 relative to neck 109) as a monostatic radar structure and hang off collar at a bottom-most position 107 relative to the animal's neck 108, closest to the trachea 110.

In another example, as shown in the configuration of FIG. 1, sensor electronics 101 are positioned at the top of the neck 108 with the antennas 103 located on the side of neck 108, also as a monostatic radar structure. Here, by placing transmit antenna 104 and receive antenna 105 closer to carotid artery 116 and jugular vein 118, the beam from the transmit antenna 104 and returning to receive antenna 105 may encounter fewer dialectically different structures when located at the position shown in FIG. 1 then when located at position 107. This reduction in the number of dialectically different structures reduces backscatter signals from those different structures.

As depicted, collar 102 may include a counterweight 106 that may be approximately the weight of antennas 103 balance the UWB radar system and attempt to maintain antennas 103 at their side placement around the neck 108.

Alternatively or in addition to counterweight 106, a tensioner may be used to maintain a relatively constant tension on collar 102 to help position antennas 103 on the side of the neck 108.

Further, as larger animals have stronger neck muscles (for instance muscles 123, 124), these muscles in some instances may form a recess 125 upward of spinal column 122. The inside shape of sensor electronics 101 may be convex to allow at least some nestling in the concave recess formed by muscle groups 123 and 124.

By placing antennas 103 and aside position as shown in FIG. 1, accurate readings from the animal's carotid artery 116 and/or jugular vein 118 may be obtained. Depending on the type of animal, the antennas 103 may be angled relative to neck 108 and/or to each other to allow for illumination of relevant structures and collection of backscattered signals from those structures. For instance, to concentrate solely on carotid artery 116, the receive antenna 105 may be moved closer to transmit antenna 104 to receive the stronger backscatter signals roughly in line with the radiated beam from transmit antenna 104 plan. Alternatively, to concentrate on carotid artery 116 and the movement of muscles 114 and 115 surrounding esophagus 113, receive antenna 105 may be moved farther away from transmit antenna 104. Further, to also include signals from the movement of muscles 111 and 112 surrounding trachea 110, the receive antenna 105 may be moved further from transmit antenna 104. In these examples, the various muscle groups may be monitored surrounding the trachea 110 as the trachea's cartilage may not reflect the UWB pulses and the movement of the cartilage is not detectable directly.

In many applications across a range of species, the UWB radar sensor can be placed within or on a collar or harness where the choice of the garment and specific sensor placement upon or within the garment is driven by the desired medical data, the need to locate the sensor in the proximity of the key primary and alternative secondary anatomical structures required to obtain the desired data, and the need to secure the sensor to the animal such that it is unlikely to be dislodged or removed during normal activity. In addition, the shape of the sensor and its antennas can be modified to take advantage of the anatomy to assist with placement and maintain position.

Actual signal processing and display of results does not have to be co-located with the sensor and in fact, remote processing and display might be highly desirable. The data can be processed (partially or completely) locally using an embedded processor (for instance, microcontroller or discrete signal processor) or wirelessly transferred to another processing platform—dedicated base station, smart phone, tablet, PC, or the cloud using a conventional wireless transfer system (transmitter in the sensor electronics 101 to transmit a signal to of receiver over, for instance a Wi-Fi connection). The display can be a digital readout on a panel built into the base station or take advantage of the GUI capabilities of any number of consumer electronics.

Of the various limitations described herein, a collar 102 allows collection of basic cardiopulmonary data without the need to be directly over the heart and lungs. The collar with its UWB radar system collects data primarily from the carotid arteries in the neck, as well as physiological data associated with motion of the larynx, trachea, and esophagus. Data from these structures enables monitoring of consumption—e.g., food and water, vomiting and regurgitation, as well as enabling the detection of choking and vocalization—e.g., barking, or other processes involving the larynx and trachea based on analyses of received signals including identification of the frequency components of the signals, the magnitude of those frequency components, and how those signals change over time. Other sensor technologies may be added to the assembly to support data fusion for improved accuracy, reliability, and noise reduction.

Further, an additional counterweight (e.g., the animal's tag or other collar attachment) may be provided at location 107 to provide a weight that may further aid in aligning the sensor electronics 101 and antennas 103.

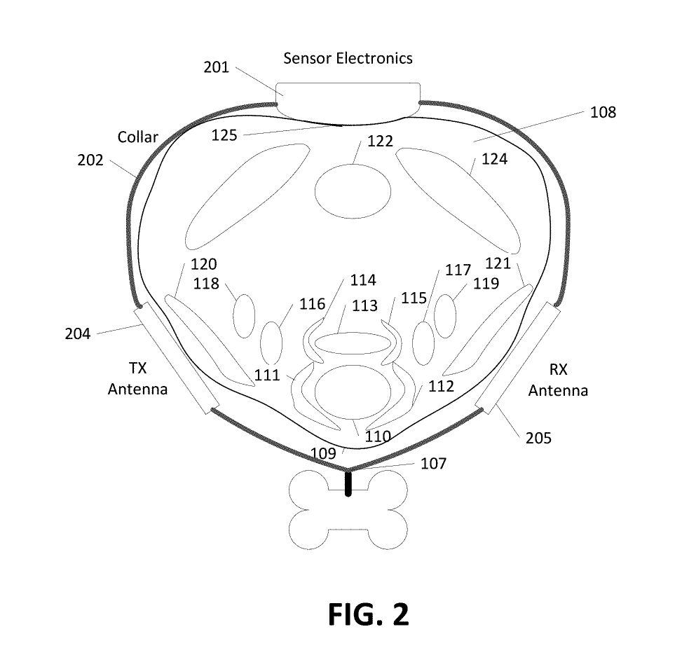

FIG. 2 shows another configuration of sensor electronics 201 and the antennas. In FIG. 2, transmit antenna 204 is located on a first side of neck 108 and receive antenna 205 is located on a symmetrically opposite side of neck 108. Here, the antennas 204 and 205 may be symmetrically distributed around the circumference of the neck 108 to maintain an even weight distribution on collar 202. One example of this configuration would have the sensor electronics 101 in the depression over the spinous process 125—see FIGS. 1 and 2, enabling easy and consistent placement of the sensor electronics 201. Unlike the anterior of the neck where many animals have a wattle and are sensitive to any object, this location typically has less fat tissue, less loose skin, and has less anatomical variation within a specific species or breed.

The configuration of transmit antenna 204 being separate from receive antenna 205 FIG. 2 is commonly referred to as a bistatic radar architecture. In the minimal separation case, both the TX and RX antennas may be located along the spine while in the limit, they could be located on either side of the larynx.

In FIG. 2, the receive antenna 205 may receive backscatter from some structures inside neck 108. For structures that have a strong dielectric difference from surrounding structures, the amplitude of the backscatter signals may predominate the received collection of signals. But for structures that have a less significant dielectric difference from surrounding structures, the resultant backscatter from these less significant dielectric differences is weaker. Thus, when attempting to monitor movement of different structures relative to each other where the dielectric constants of these structures are relatively close to one another, monitoring backscatter signals is more difficult. In this situation, monitoring signal modification (signal amplification, attenuation, polarization, retardation or advancement, and the like) with a receive antenna 205 generally facing transmit antenna 204 is preferable.

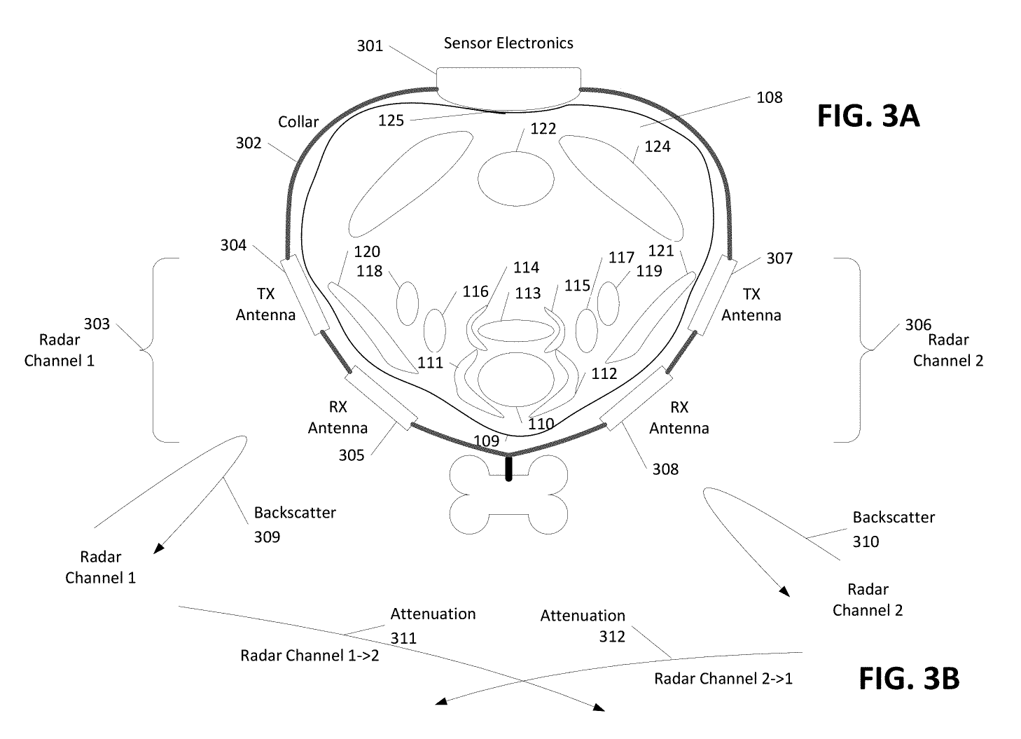

The above bistatic of FIG. 2, configuration could be expanded to a multistatic configuration with a corresponding increase in weight, cost of goods, and power consumption. As shown in FIG. 3A, the sensor may include two radar channels 303 and 306, each consisting of a TX and RX pair (304/305 and 307/308, respectively), where one radar channel interrogates the right side of the neck and one radar channel interrogates the left side.

This configuration takes advantage of the symmetry in the neck to improve signal reception while reducing common noise. More radar channels may be added for additional performance improvements.

As shown in FIG. 3B, radar channel 1 303 is shown on the left side of FIG. 3A and radar channel 2 306 is shown on the right side of FIG. 3A. The backscatter signal 309 of radar channel 1 303 from transmit antenna 304 enters and then returns back through the side of neck 108 to receive antenna 305. Similarly, the backscatter signal 310 of radar channel 2 306 from transmit antenna 307 enters and then returns back through side 306 of neck 108 to receive antenna 308. Also, receive antenna 308 receives attenuation signal 311 (from radar channel 1 to radar channel 2) as originally transmitted from transmit antenna 304. Likewise, receive antenna 305 receives attenuation signal 312 (from radar channel 2 to radar channel 1) as originally transmitted from transmit antenna 307.

To permit attenuation signals 311 and 312 to be received and used by sensor electronics 301, common timing signals used to control the transmission of the UWB pulses in the multistatic UWB radar system are used in radar channel 1 and radar channel 2. For instance, when transmit antenna 304 has finished transmitting, both receive antenna 305 and receive antenna 308 may both be active (in accordance with the same or a temporally adjusted timing signal) in receiving signals scattered and/or modified by the combination of various structures in neck 108. Alternatively, transmit antenna 304 and transmit antenna 307 may transmit simultaneously in accordance with the same or temporally adjusted timing signal with one of receive antenna 305 or receive antenna 308 also being active (and likewise being responsive to the same or temporally adjusted timing signal). Finally, transmit antenna 304 and transmit antenna 307 may both transmit simultaneously and receive antenna 305 and receive antenna 308 may both receive signals simultaneously with all operations coordinated through the same or temporally adjusted timing signal. The purpose using the same or temporally adjusted timing signal in sensor electronics 301 is to provide temporal coherence for the operations of radar channel 1 303 and radar channel 2 306.

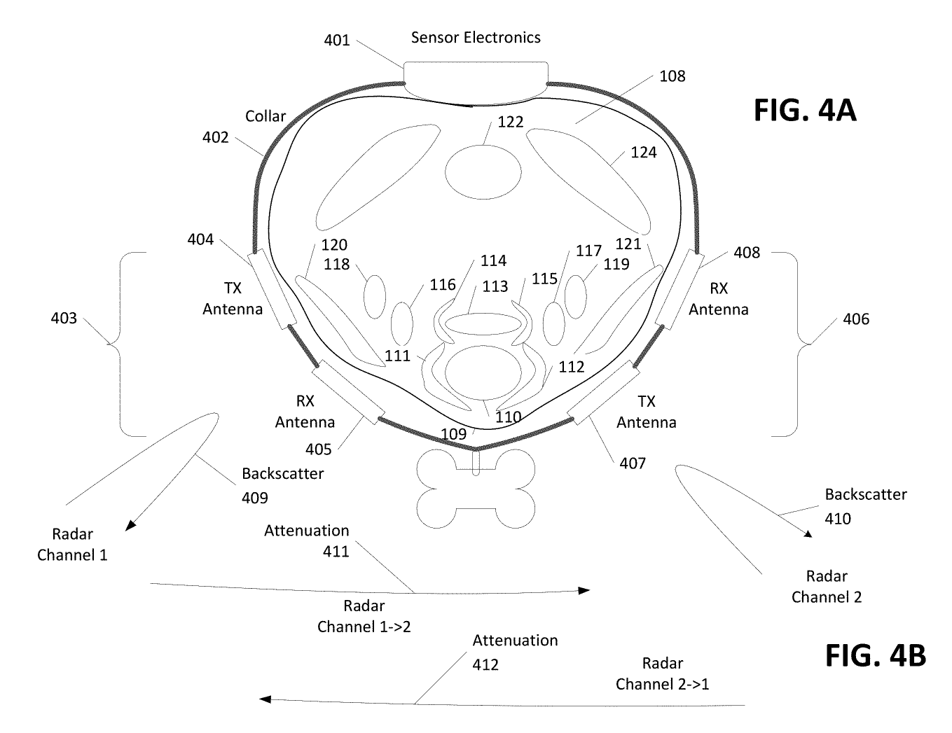

FIG. 4A shows a similar structure to that of FIG. 3A in which sensor electronics 401 controls radar channel 1 403 (with transmit antenna 404 and receive antenna 405) and greater channel 2 406 (with transmit antenna 407 and receive antenna 408). Here, the locations of the transmit antenna and receive antenna of radar channel 2 406 are flipped relative to the locations of transmit antenna 404 and receive antenna 405. While backscatter signal 409 of radar channel 1 is similar to that shown in FIG. 3B, backscatter signal 410 is reflected more upwards then backscatter signal 310 of FIG. 3B (which is reflected more downwards). Also, attenuation signal 411 from transmit antenna 404 to receive antenna 408 is generally more horizontal than attenuation signal 311. Similarly, attenuation signal 412 from transmit antenna 4072 receive antenna 405 is also generally more horizontal than attenuation signal 412.

As with the sensor electronics 301 of FIG. 3A, sensor electronics 401 of FIG. 4A may also use temporally coherent timing signals to allow the multistatic operation of the transmit and receive antenna components of FIG. 4A.

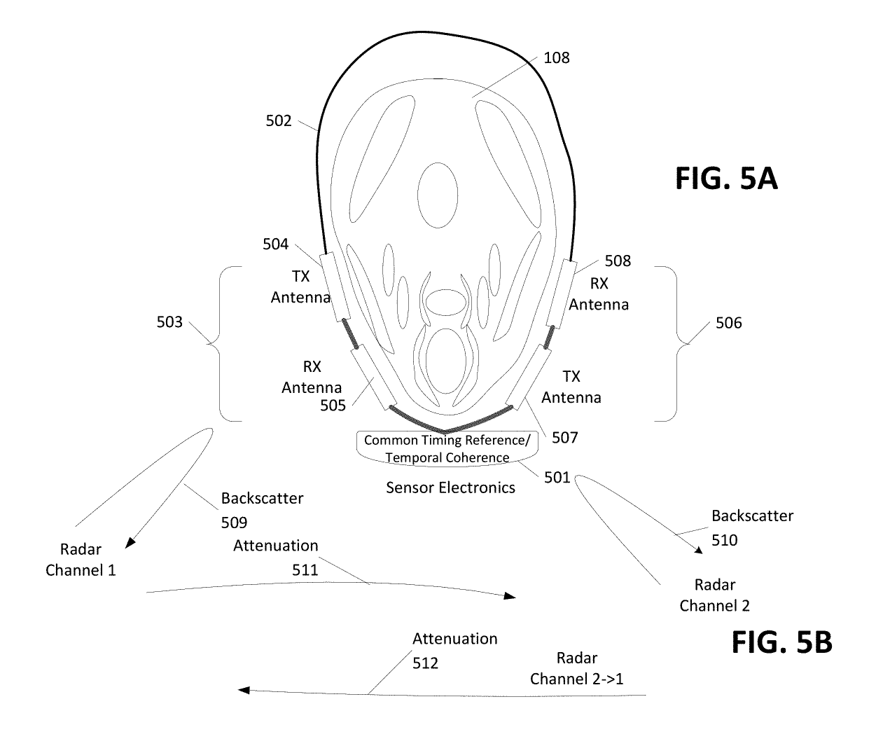

FIG. 5A shows a configuration similar to that of FIG. 4A but with an animal having a narrower neck 108. FIG. 5A shows collar 502, sensor electronics 501 (with the common timing reference providing temporal coherence among radar channel 1 503 and radar channel 2 506), transmit antennas 504 and 507, receive antennas 505 and 508. FIG. 5B shows backscatter signals 509 and 510 and attenuation signals 511 and 512.

In all cases (including monstatic, bistatic, and multistatic), the location, orientation, and antenna characteristics of the paired TX and RX antennas for each radar channel may be designed to allow convergence of the TX and RX antenna bore sights onto the anatomical structure of interest while maintaining sufficient beamwidth at the structure of interest.

As described above, a counterweight may be integrated to minimize the potential for collar rotation while a tensioning device (springs or clips or elastically deformable materials) may be added to maintain constant pressure against the animal's neck 108, minimizing the noise caused by motion at the sensor/skin interface. Also, it is important to note that the sensor electronics and antennas do not need to be co-located as the electronics can connect to the antennas via cables or flexible circuit boards. Either of these connection techniques can be embedded into the collar itself as long as the connecting media is relatively homogeneous to minimize RF reflections.

A harness—e.g., a modified walking harness, has the advantage of allowing one or more radars to interrogate various anatomical regions of interest or to enable more sophisticated signal processing by isolating on a particular organ. For example, if the UWB radar sensor has at least one channel proximal to the heart, advanced cardiac biometrics can be obtained, including stroke volume, cardiac output, and changes in blood pressure. Similarly, if the UWB radar sensor has one channel proximal to main right and left nodes of the lungs, the system can check for asymmetrical breathing patterns.

The UWB radar is not limited to the torso for collecting cardiopulmonary data as there are many alternative locations on the animal that can be exploited, particularly for obtaining cardiac data. For example, good quality cardiac data can be collected by positioning the UWB sensor in proximity of the carotid arteries to take advantage of the expansion and contraction in the radius of the arteries throughout the cardiac cycle. In addition, positioning the sensor on the neck has been shown to provide reasonable and quantifiable respiratory information.

Various porcine animal models (e.g., weights between 30-50 kg) have been studied to develop new human cardiopulmonary monitoring systems. In these studies, a UWB radar sensor was placed to the left of the animal's sternum, proximal to the heart and collected cardiopulmonary data in parallel with other reference monitors. Data from the UWB radar sensor was processed with proprietary signal processing algorithms and the results correlated against the data from the reference monitors to determine the efficacy of the radar sensor. The UWB sensor demonstrated the ability to measure cardiac and pulmonary rate, detect changes in cardiac stroke volume, measure CPR compressions, and determine the status of the circulatory system across a variety of cardiac conditions.

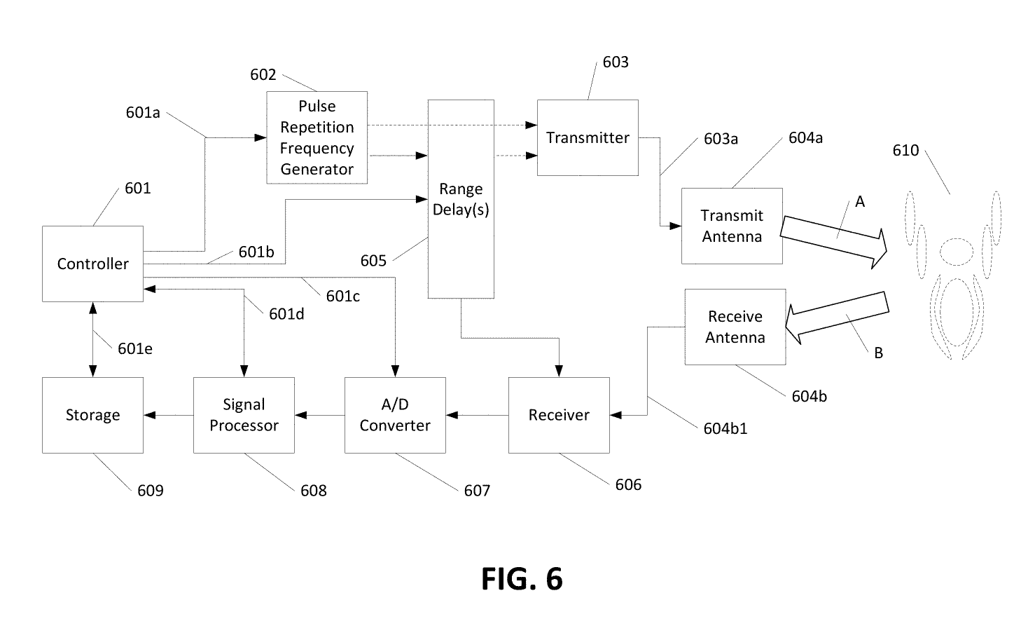

FIG. 6 shows a conventional configuration for a UWB radar system as known in the art. The UWB radar system of U.S. Pat. No. 7,725,150 is incorporated herein by reference. The controller 601 generates the timing and control signals 601a, 601b, 601c, 601d, and 601e to synchronize and manage the rest of the system. It also accepts internal feedback signals from the other subsystems, accepts external control inputs from an operator, and has the capability of providing data outputs to the operator or medical record system. The controller can be realized using an integrated processor and associated circuitry.

Based on timing and control signals 601a from the controller 601, the pulse repetition frequency (PRF) generator 602 creates the baseband pulse train used by the transmitter 603 and, after range delay Δt 605, by the receiver 606. Alternately, both the transmitter 603 and the receiver 606 may receive a delayed signal from the pulse repetition frequency generator 602. Further, the delay applied to either or both of the transmitter 603 and the receiver 606 may be fixed or variable.

Since the pulse train is common to both the transmitter and receiver subsystems and allows them to operate synchronously, the system is a time-coherent radar system. In practice, a voltage-controlled oscillator (VCO) operating at a nominal but only exemplary output frequency of 2 MHz in or associated with the PRF generator supplies the pulse train. Randomized pulse-to-pulse dither can be added to the output of generator 2 by injecting a noise signal from a noise signal source (not shown) into the VCO control port. The random dither causes spectral spreading to reduce the probability of interfering with other electronic devices as well as provide a unique transmit coding pattern per unit, allowing multiple units to operate in close proximity without substantial concern for mutual interference.

Transmitter 603 generates a series of low-voltage, short-duration pulses 603a (in one embodiment, less than 200 ps) based on the pulse train from the PRF generator 602. In practice, differentiating the edges of a pulse train having extremely fast rising and falling edges creates the sub-nanosecond pulses. Through the combination of the transmitter and the antenna, the short duration pulses are converted into an ultra-wide band spectrum signal centered in the RF/microwave frequency bands in accordance with FCC R&O 02-48.

In one embodiment, the transmitter 603 and receiver 606 share a common antenna 604. In another embodiment, the antennas are separated into transmit antenna 604a and receive antenna 604b. For the transmitter, the antenna 604a couples the short pulses from the transmitter 603 to the environment, as illustrated at A, to a patient. Subsequently, reflections B are received from the environment and fed to the receiver 606. Various antenna configurations may be used including: commercially available horns and flat resonators, simple magnetic dipoles, and a magnetic dipole or “loop” antenna(s) with a diameter selected to optimize the transmission and reception of UWB signals. For example, a loop antenna with a diameter of 4 cm fabricated from 24-gauge solid copper wire was used in conjunction with a UWB system operating with a 10 dB bandwidth of 1.5 Ghz to 3.4 Ghz.

Based on timing and control signals 601b from the controller 601 and the pulses originating from the PRF generator 602, the range delay Δt 605 generates a delayed version of the PRF timing signal. The output of the range delay triggers a sample-and-hold circuit, described subsequently, in the receiver 606 where the delay value is chosen to compensate for fixed electrical delays within the system and focus data collection to those reflections originating from a specific depth within the body. The range delay is extremely flexible and, in conjunction with the controller, can generate a large range of delay profiles to accommodate a variety of signal processing requirements.

There are two delay modes used to collect medical data—range gate mode and range finder mode. In range gate mode, the depth within the body that corresponds to the area for which physiological data is to be extracted is fixed and a large number of samples are collected at that depth over a period of multiple seconds in one example, providing information on relative changes within the body. The depth can then be changed and the process repeated. In contrast, when operating in range finder mode, the depth is swept repeatedly over a finite range of interest, with samples collected at each depth. Range gate mode provides detailed information at the depth of interest while range finder mode is used to quickly collect data over a range of depths. A range delay circuit supports both range gate and range finder modes. In practice, the range delay circuit can be realized using a 12-bit digital-to-analog converter (DAC), an operational amplifier, used to realize functions, and a one-shot multivibrator. The one-shot multivibrator (an LMC555 can be used, as one example) generates a delayed version of the transmitted pulse train in response to signals received on its two control inputs—trigger and hold-off. The pulse train from the PRF generator 602 is the trigger signal and causes the one-shot multivibrator to initiate a single pulse cycle for each pulse in the pulse train. The hold-off voltage determines the period of the pulse. By varying the hold-off voltage, different pulse periods, and thus different delay values, can be generated. The amount of delay is set by both analog and digital controls. The analog controls set the minimum delay value and the allowable range of control while the digital controls are used to dynamically adjust the actual delay value, delay sweep rate, and resolution of delay control.

In practice, a 12-bit data value—Dataₓ, corresponding to the desired delay, is sent from the controller 601 to the DAC. The DAC produces a voltage V, where:

$${V}_{x} = 4.096 Volts \times \frac{{Data}_{x}}{4096} .$$

The DAC output voltage and a DC voltage are added together in a summing junction and the sum is amplified and fed to the hold-off control input of the one shot. The DC voltage level, in conjunction with the amplifier gain, set the minimum delay value and the allowable range of control. Both the DC voltage level and gain settings are controlled by manual adjustment of potentiometers. A delay range of 5 ns has been proven to yield good quantitative data in cardiopulmonary applications and corresponds to a depth range of approximately 12 cm into the body. Other delay range values of up to 10 ns have also shown to produce usable data sets.

The receiver 606 processes the raw reflections received from the antennas 604b over line 604b1 in the analog domain to optimize the signals of interest. For cardiopulmonary data, this includes suppressing the high-strength static return signals and amplifying the motion artifacts. Receiver 606 may be based on a dual-channel balanced receiver architecture where the transmitter pulses are capacitively coupled from the output of the transmitter 603 into both receive channels via RF. Splitter and the antenna 604 is connected or otherwise coupled to one channel. The balanced receiver architecture provides a high degree of common mode rejection as well as differential gain. The common mode rejection provides a significant amount of attenuation to signals common to both channels thus minimizing interference from the transmit signal with the desired receive signal. The differential gain inherent in this architecture amplifies signals unique to either channel thus the received signal, being unique to the channel, is amplified.

Both channels can use an ultra-fast sample-and-hold (S/H) circuit, each triggered by the delayed impulse train created by the pulse generator using the delayed pulse train over the line from the range delay circuit Δt 5 of FIG. 6. The active sampling window is set at approximately 320 ps in one example and can be easily modified by selectively changing the value of a single passive component. The outputs of the two S/H circuits are integrated over multiple samples in integrator elements to improve the signal-to-noise ratio. The integrated samples feed the inverting and non-inverting inputs of an instrumentation amplifier, attenuating the transmitted signal and amplifying the received signal.

As illustrated in FIG. 6, A/D converter 607 (ADC) is controlled by controller 601 through control lines 601c. The controller sets the sample rate, sample resolution, and start/stop timing for the sampling process based on the mode of operation. The ADC digitizes the enhanced analog motion reflections from the receiver 606, translating the enhanced reflected energy into a series of discrete digital values. As one example in range gate mode, 16,000 samples per second at 16-bits per sample may be used.

The digitized signal from the A/D converter 607 is then processed to extract pertinent physiological information in signal processor 608 per FIG. 6. The signal processing block is extremely flexible and, as mentioned previously, can accommodate a wide variety of algorithms in support of different medical applications. In addition the algorithm can be implemented using parallel, serial, or hybrid parallel/serial architecture. The choice of a specific architecture is left to those skilled in the art and will depend on the application and other system constraints. The controller manages the signal processing operations through control path 601d.

The resultant physiological data is displayed on a user interface (not shown). This can include tracings of amplitude versus time for one or more depths of interest, power spectral density for one or more depths of interest, time domain and frequency domain histograms for a range of depths, numerical values for heart and/or lung rates, as well as the associated confidence factors for the displayed data, as described subsequently. The controller 601 of FIG. 6 converts the data from the signal processor to an operator-friendly format through control path 601e for display on the user interface.

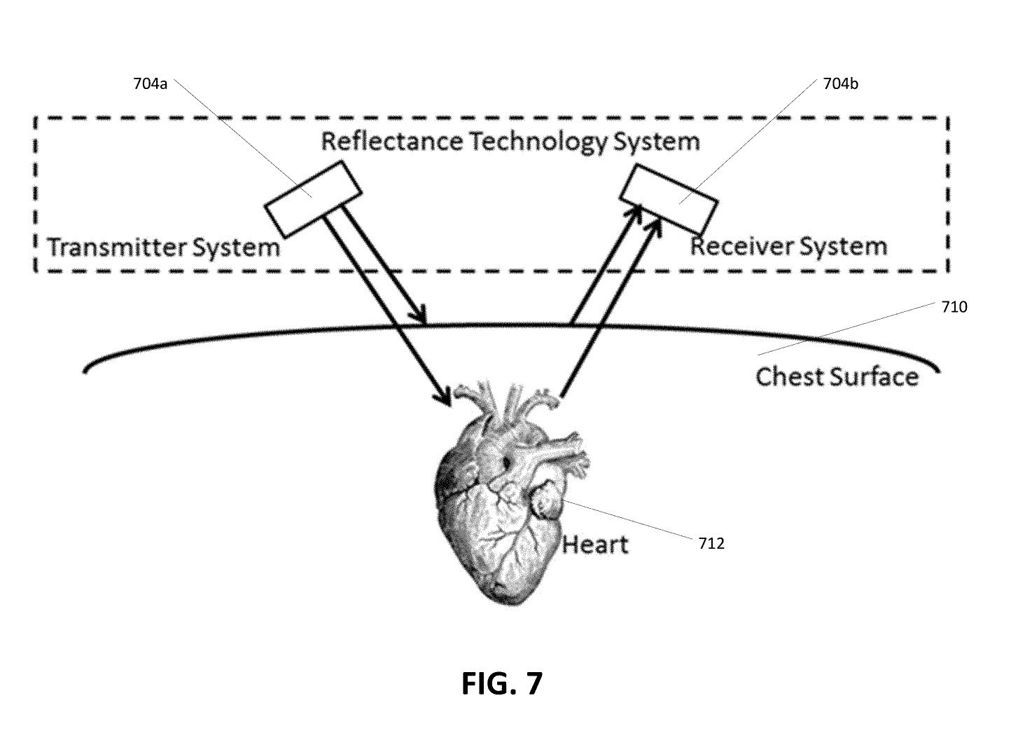

FIG. 7 depicts an illustrative reflectance technology system. A reflectance technology system may use IR, UWB, or reflectance technology, or a combination of reflectance technologies (e.g., IR-UWB). The reflectance technology system may include one or more transmitters and one or more receivers. The transmitter may be configured to transmit a signal (e.g., an IR-UWB radar signal). The transmitted signal 704a may penetrate a chest surface and enter a body 710. The transmitted signal may reflect off a heart 712 or another organ. The receiver may be configured to receive the reflected signal 704b (e.g., the IR-UWB radar signal). The transmitter and/or receiver may be situated in a single housing, or may be situated in different housings. The transmitter and/or receiver may be at an angle relative to each other, such that the transmitted signal is configured to be directed at the heart 712 and reflect back to the receiver.

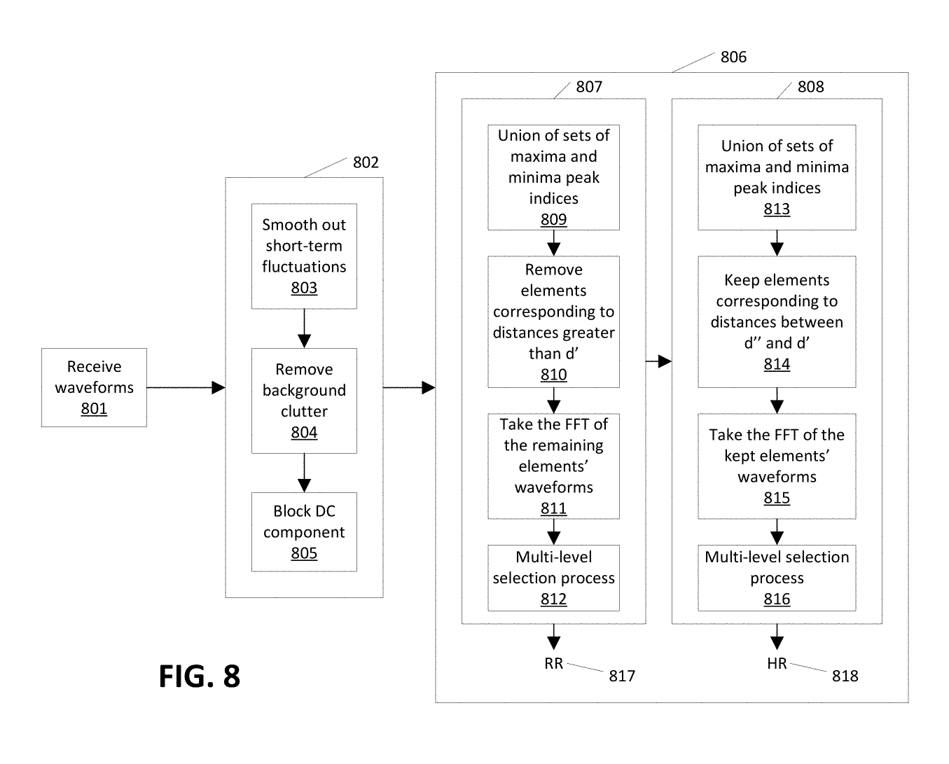

FIG. 8 depicts an illustrative block diagram of a process for using reflectance technology to determine one or more vital signs. One or more vital signs, such as cardiopulmonary vital signs (e.g., respiration, heart rate), may be inferred based on the processing of recorded waveforms that are collected by an IR-UWB radar, and/or any other reflectance technology system, after being reflected-off a subject's body. A base rate may be inferred.

The procedure through which the recorded waveforms are processed in order to extract these vitals is described below. The procedure may include one or more phases (e.g., three phases). A phase may include noise reduction. A phase may include respiration rate extraction. A phase may include heart rate extraction. One or more of the aforementioned phases may use tools and/or techniques from signal processing and/or logic analysis. This procedure may tackle conditions of very high respiration rate and very low heart rates. This procedure may suppress the effects of harmonics and intermodulation between the breathing and heartbeat signals. In some instances, this procedure might not require any filter implementation.

A phase 802 (e.g., a first phase) may include noise reduction. One or more inputs to the noise reduction phase may include received waveforms 801 and/or D.

In step 803, the system may smooth out short-term fluctuations and highlight longer-term trends by applying a k-point moving average →R.

In step 804, the system may remove background clutter by subtracting the average of all waveforms in R from each signal in R→X.

In step 805, the system may block a DC component by subtracting the average of all columns in X from each column in X→Y.

A phase 807 (e.g., a second phase) may include Respiration Rate Estimation. One or more inputs to the respiration rate estimation phase may include Y.

In step 809, the system may find U (e.g., a set that is the union of two sets that list the maxima and minima peaks' indices). More formally, the system may record the index of maximum and/or minimum peaks of every received waveform in two or more different sets. Then, the system may take the union of these two sets whose elements are highly likely, in some instances, to contain the breathing signal.

In step 810, the system may remove the elements in U that have a propagation time or distance greater than

$$\frac{{t}^{′}}{2}$$

or d′ respectively, according to the following equation:

$${d}^{′} = \frac{V \times {t}^{′}}{2} = \frac{C}{\sqrt{{ɛ}_{r}}} \times \frac{{t}^{′}}{2}$$

where C is the speed of light, where εᵣ is the relative permittivity of the medium in which signals are propagating, and where t′ is the round-trip propagation time

$$( e . g . , 2 \times \frac{{t}^{′}}{2} = {t}^{′} ) .$$

One potential statistical scenario may include where εᵣ=50 when the signal propagates through the subject's body. In another example, for respiration rates, chest displacements may be between a particular range (e.g., between 0.1 mm and several millimeters).

In step 811, the system may take the Fast-Fourier Transform (FFT) of the received waveforms with column indices that remained as elements in U, with time of flight less than t′.

In step 812, the system may record the index of each frequency at which the power spectral density (PSD) admitted a maximum for each of the elements that passed through the FFT.

The system may keep the set of unique indices along with their number of occurrences and maximum PSD value.

In step 817, the system may then choose respiration rate (RR) to be the frequency with the highest number of occurrences; if there is a tie, the system may choose RR to be the frequency that admits the maximum PSD; if there is still a tie, the system may choose RR to be the frequency that has the highest number of maximum PSD values. Note that the order of applying the last two measures can be swapped.

A phase 808 (e.g., a third phase) may include Heart Rate Estimation. One or more inputs to the heart rate estimation phase may include Y and/or RR.

In step 813, the system may find U (e.g., a set which is the union of two sets that list the maxima and minima peaks' indices). More formally, the system may record the index of maximum and minimum peaks of every received waveform in two different sets. Then, the system may take the union of these two sets whose elements are highly likely to contain the breathing signal.

In step 814, the system may remove the elements in U that that has a propagation time or distance greater than

$$\frac{{t}^{′}}{2}$$

or d′ respectively, and less than

$$\frac{{t}^{″}}{2}$$

or d″, respectively. Here, the relation between t″ and d″ are similar to the relation between t′ and d′, as shown in the aforementioned equation in the second phase. Note that, for heart rates, typical chest displacements are less than a threshold (e.g., 0.08 mm).

In step 815, the system may take the FFT of the received waveforms with column indices being the remaining elements with time of flight between t″ and t′.

The system may record the index of each frequency at which the PSD admitted a maximum for each of the elements that passed through the FFT.

The system may keep the set of unique indices along with their number of occurrences and the variance of PSD values.

The system may translate the frequency indices into their corresponding heart frequency values and group them into segments.

In step 816, the system may select the segment that has the maximum number of heart frequencies in its range; if there is a tie, the system may select the segment that has the largest sum of PSD values.

The system may choose HR to be the frequency with the highest number of occurrences in the selected segment; if there is a tie, the system may choose HR to be the frequency that admits the minimum variance among its PSD values; if there is a tie, the system may choose HR to be the frequency that has the highest number of maximum PSD values. Note that the order of applying the last two measures can be swapped

The system may check if HR is an integer multiple of RR; if no, HR is obtained 818; if yes, HR and one of RR's harmonics perfectly overlap.

One or more embodiments may be implanted in a method, apparatus, or system. Computer-readable media or memory may store executable instructions that, when executed by one or more processors, cause an apparatus or system to perform one or more steps herein. Steps may be performed in any order, repeated, omitted, or the like.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps illustrated in the illustrative figures may be performed in other than the recited order, and one or more depicted steps may be optional in accordance with aspects of the disclosure.

What is claimed is:

1. A collar comprising:
a transmitter configured to transmit an ultra-wideband signal;
a receiver configured to receive the ultra-wideband signal;
a processor; and
memory storing executable instructions that, when executed by the processor, cause the collar to:
receive one or more waveforms of the ultra-wideband signal from the receiver;
apply a k-point moving average to the one or more waveforms to obtain one or more signals;
subtract an average of all waveforms from each signal of the one or more signals;
block a DC component of the one or more signals;
determine a heart rate or respiratory rate from the one or more signals; and
send the determined heart rate or respiratory rate to a different device.

2. A method comprising:
receiving, by a processor of a collar, signals from an ultra-wideband receiver of the collar;
creating, by the processor of the collar, a first set of maximum peak indices from the signals;
creating, by the processor of the collar, a second set of minimum peak indices from the signals;
creating, by the processor of the collar, a union set that is the union of the first set and the second set;
removing, by the processor of the collar, elements in the union set that have a propagation time t or distance d greater than
$$\frac{{t}^{′}}{2}$$
or d′,
where
$${d}^{′} = \frac{V \times {t}^{′}}{2} = \frac{C}{\sqrt{{ɛ}_{r}}} \times \frac{{t}^{′}}{2} ,$$
where C is the speed of light, where εᵣ is the relative permittivity of the medium in which signals are propagating, and where t′ is a round-trip propagation time;
obtaining, by the processor of the collar, a Fast-Fourier Transform of the received waveforms with column indices that remained as elements in U, with a time of flight less than t′;
recording, by the processor of the collar, the index of each frequency at which a power spectral density (PSD) admitted a maximum PSD for each of the elements that passed through the Fast-Fourier Transform;
recording, by the processor of the collar, a set of unique indices with a number of occurrences of the unique indices and the maximum PSD; and
selecting, by the processor of the collar, a respiration rate to be a frequency with a highest number of occurrences.

3. The method according to claim 2, further comprising:
based on a tie, selecting, by the processor of the collar, a respiration rate to be a frequency that admits the maximum PSD.

4. The method according to claim 3, further comprising:
based on the tie continuing to exist, selecting, by the processor of the collar, a respiration rate to be a frequency that has a highest number of maximum PSD values.

5. The method according to claim 2, further comprising:
based on a tie, selecting, by the processor of the collar, a respiration rate to be a frequency that has a highest number of maximum PSD values.

6. The method according to claim 5, further comprising:
based on the tie continuing to exist, selecting, by the processor of the collar, a respiration rate to be a frequency that admits the maximum PSD.

7. A method comprising:
receiving, by a processor of a collar, signals from an ultra-wideband receiver of the collar;
using, by the processor of the collar, the signals to create a first set of maximum peak indices;
using, by the processor of the collar, the signals to create a second set of minimum peak indices;
creating, by the processor of the collar, a union set that is the union of the first set and the second set;
removing, by the processor of the collar, elements from the union set that have a propagation time or distance greater than
$$\frac{{t}^{′}}{2}$$
or d′ and less than
$$\frac{{t}^{″}}{2}$$
or d″, respectively,
where
$${d}^{′} = \frac{V \times {t}^{′}}{2} = \frac{C}{\sqrt{{ɛ}_{r}}} \times \frac{{t}^{′}}{2} , \\ {d}^{″} = \frac{V \times {t}^{″}}{2} = \frac{C}{\sqrt{{ɛ}_{r}}} \times \frac{{t}^{″}}{2} ,$$
where C is the speed of light, and where εᵣ is the relative permittivity of the medium in which signals are propagating;
obtaining, by the processor of the collar, a Fast-Fourier Transform of the received waveforms with column indices that remained as elements in U, with a time of flight between t″ and t′;
recording, by the processor of the collar, an index of each frequency at which a power spectral density (PSD) admitted a maximum PSD for each of the elements that passed through the Fast-Fourier Transform;
recording, by the processor of the collar, a set of unique indices with a number of occurrences and the maximum PSD;
selecting, by the processor of the collar, a respiration rate to be the frequency with the highest number of occurrences;
translating, by the processor of the collar, the frequency indices into corresponding heart frequency values;
grouping, by the processor of the collar, the frequency indices into segments;
selecting, by the processor of the collar, a segment of the segments that has a maximum number of heart frequencies in a range of the segment;
selecting, by the processor of the collar, a heart rate to be the frequency with the highest number of occurrences in the selected segment;
determining, by the processor of the collar, whether the selected heart rate is an integer multiple of RR;
based on determining that the selected heart rate is not the integer multiple of RR, obtaining, by the processor of the collar, a heart rate; and
based on determining that the selected heart rate is the integer multiple of RR, determining, by the processor of the collar, an overlap between the heart rate and a harmonic of the respiration rate.

8. The method according to claim 7, further comprising:
based on determining a tie, selecting, by the processor of the collar, the heart rate to be a frequency that admits the minimum variance among PSD values of the heart rate.

9. The method according to claim 8, further comprising:
based on determining that the tie still exists, selecting, by the processor of the collar, the heart rate to be a frequency that has a highest number of maximum PSD values.

10. The method according to claim 7, further comprising:
based on determining a tie, selecting, by the processor of the collar, the heart rate to be the frequency that has a highest number of maximum PSD values.

11. The method according to claim 10, further comprising:
based on determining that the tie still exists, selecting, by the processor of the collar, the heart rate to be a frequency that admits a minimum variance among PSD values of the heart rate.
